(19) United States
(12) Patent Application Publication
(10) Pub. No.: US 2013/0034611 A1
(43) Pub. Date: Feb. 7, 2013

(54) SUSTAINED PREPARATION OF FACTOR IX

(71) Applicants: Li-Chien CHANG, Taipei (TW); Chi-Yu YANG, Kaohsiung (TW); Yi-Juain LIN, Miaoli (TW)

(72) Inventors: Li-Chien CHANG, Taipei (TW); Chi-Yu YANG, Kaohsiung (TW); Yi-Juain LIN, Miaoli (TW)

(73) Assignee: ANIMAL TECHNOLOGY INSTITUTE TAIWAN, Miaoli Hsien (TW)

(21) Appl. No.: 13/554,274

(22) Filed: Jul. 20, 2012

Related U.S. Application Data
(63) Continuation of application No. PCT/CN2011/000102, filed on Jan. 21, 2011.

(30) Foreign Application Priority Data
Jan. 21, 2010 (EP) 10151282.0

(51) Int. Cl. A61K 9/14; A61P 7/04; A61K 38/36

(52) U.S. Cl. 424/497; 514/13.7; 424/490

(57) ABSTRACT

The present invention provides a pharmaceutical preparation in powder-like form, comprising a therapeutically effective amount of a human Factor IX (hFIX) encapsulated by a lipophilic biodegradable polymer or copolymer to form a microsphere, whereby the pharmaceutical preparation provides a sustained release of hFIX and a prolonged biological activity.

### FIELD OF THE INVENTION

This invention relates to a sustained preparation of encapsulated Factor IX.

### BACKGROUND OF THE INVENTION

Factor IX is one of the serine proteases of the coagulation system in the body. Deficiency of such protein causes Hemophilia B, a blood clotting disorder, which is also known as Christmas disease. Patients with hemophilia B are more prone to bleeding than normal subject and have poor wound healing after injury or surgery. Bleeding generally happens at the muscles or the spaces of joints, such as elbows, knees and ankles, and also happens at the brain and the spinal cord, the throat or the gut. Hemophilia B is a lifelong disease that is life threatening.

Conventionally, the treatment for hemophilia B patients is intravenous injection of condensed factor IX, e.g., commercially available BeneFIX® (Wyeth), to replace the missing or mutated protein. However, polypeptide drugs are rapidly degraded by proteolytic enzymes or neutralized by antibodies, and thus their half-life and circulation time are reduced, thereby limiting their therapeutic effectiveness.

PEGylation of proteins is a well-established technique in protein chemistry to enhance protection of protein drugs from proteolytic degradation. For example, PCT International Application WO 2009/083187A1 provided a chemically modified blood coagulation factor IX (Factor IX) comprising a Factor IX activation peptide region (AP region), wherein said AP region comprises a covalently coupled water-soluble hydrophilic polymer. In one preferred embodiment of the PCT application, the water-soluble hydrophilic polymer was attached to Factor IX via Asn-157 and/or Asn-167 of Factor IX. In an alternatively preferred embodiment, the water-soluble hydrophilic polymer was attached to Factor IX via Ser-158, Thr-159, Thr-163, Thr-169, Ser-171, Thr-172, Ser-174 or Thr-179, especially via Ser-158, Thr-163, Ser-171 or Ser-174 of Factor IX, and generally the water-soluble hydrophilic polymer is PEG. However, adding PEG chemically as such would inevitably change the structure of the polypeptide and might cause unexpected side effects.

### SUMMARY OF THE INVENTION

The present invention features a new pharmaceutical preparation in a powder-like form, comprising a human Factor IX encapsulated by a lipophilic biodegradable polymer or copolymer to form a microsphere, which provides a prolonged release of human Factor IX, and is free of any remnant organic solution after lyophilization.

In one aspect, the present invention provide a pharmaceutical preparation in a powder-like form, comprising a therapeutically effective amount of a human Factor IX (hFIX), which is encapsulated by a lipophilic biodegradable polymer or copolymer to form a microsphere, whereby the pharmaceutical preparation provides a sustained release of hFIX and a prolonged biological activity, wherein the lipophilic biodegradable polymer or copolymer is selected from the group consisting of a phospholipid, a lecithin, a polyglycolic acid (PGA), a poly(lactic-co-glycolic acid) (PLGA), a poly(γ-glutamic acid), a polyvinylic acid (PVA), a γ-polyglutamic acid, a polycaprolactone, a polyanhydrides, a polyamino acid, a polydioxanone, a polyhydroxybutyrate, a polyphosphazenes, a polyesterurethane, a polycarboxsyphenoxypropane-cosebacic acid, a polyorthoester, and a combination thereof.

In the other aspect, the present invention provides a method for manufacturing the pharmaceutical preparation of the present invention comprising:
a) mixing a therapeutically effective amount of a human Factor IX (hFIX) in an aqueous solution with a lipophilic biodegradable polymer or copolymer in an organic solution to obtain a primary emulsion;
b) mixing the primary emulsion with a surfactant solution to obtain a secondary emulsion; and
c) evaporating the organic solvent, and then filtrating, washing, and lyophilizing the secondary emulsion to obtain a powder-like pharmaceutical preparation;
whereby in the power-like pharmaceutical preparation, the hFIX is encapsulated by the lipophilic biodegradable polymer or copolymer to form a microsphere, which has an efficacy of sustained release of hFIX and prolonged biological activity.

The details of one or more embodiments of the invention are set forth in the description below. Other features or advantages of the present invention will be apparent from the following drawings and detailed description of several embodiments, and also from the appending claims.

### DESCRIPTION OF THE DRAWINGS

The above aspects and advantages of the present invention will become more readily apparent in view of the drawings.

In the drawings:

FIG. 1 is a diagram showing the hFIX activities of the pharmaceutical preparation according to the present invention, which were determined by an aPTT assay at different times for the preparations of Batch 3 (PLGA75/25, 4.6 IU/mg) and Batch 4 (PLGA85/15, 4.6 IU/mg).

### DETAILED DESCRIPTION OF THE INVENTION

The detailed description of the present invention is as follows. In the present invention, references are cited in Examples. For better understanding of the present invention, technical terms used herein are described in detail separately.

Unless defined otherwise, all technical and scientific terms used herein have the meaning commonly understood by a person skilled in the art to which this invention belongs.

The articles “a” and “an” are used herein to refer to one or more than one (i.e., at least one) of the grammatical object of the article.

The invention provides a pharmaceutical preparation in a powder-like form comprising a therapeutically effective amount of a human Factor IX (hFIX), which is encapsulated by a lipophilic biodegradable polymer or copolymer to form a microsphere, whereby the pharmaceutical preparation provides a sustained release of hFIX and a prolonged biological activity. The hFIX may be naturally occurring or recombinant Factor IX. According to the invention, the pharmaceutical preparation is used for treating hemophilia B.

The term “treating” as used herein refers to curing, ameliorating or meliorating a bleeding disorder of the subject.

The term “microsphere” as used herein refers to a small spherical particle with diameters in the micrometer range. In one embodiment of the invention, the microsphere has a diameter of 0.1 to 100 μm. The microspheres of the present invention may be further lyophilized to a powder-like form for easy transportation, manipulation, and administration.

The term “biodegradable polymer or copolymer” as used herein refers to a polymer or copolymer, which may be degraded or eroded in vivo by enzymatic, chemical and/or physical processes, to form smaller chemical species.

The term “lipophilic” as used herein refers to a biodegradable polymer or copolymer being dissolvable in fats, oils, lipids, and organic solvents. In the present invention, the lipophilic biodegradable polymer or copolymer is selected from the group consisting of a phospholipid, a lecithin, a polyglycolic acid (PGA), a poly(lactic-co-glycolic acid) (PLGA), a poly(γ-glutamic acid), a polyvinylic acid (PVA), a γ-polyglutamic acid, a polycaprolactone, a polyanhydrides, a polyamino acid, a polydioxanone, a polyhydroxybutyrate, a polyphosphazenes, a polyesterurethane, a polycarboxsyphenoxypropane-cosebacic acid, a polyorthoester, and a combination thereof. In one example of the invention, the lipophilic biodegradable polymer or copolymer is PLGA, for example in a concentration of approximately 1 mg/ml to 1000 mg/ml, preferably 90 mg/ml.

Depending on the ratio of lactide to glycolide used for the polymerization, different forms of PLGA may be obtained: these are usually expressed in regard to the monomers' ratio used (e.g. PLGA 75:25 indicates a copolymer whose composition is 75% lactic acid and 25% glycolic acid). According to the present invention, the monomers' ratio is PLGA 10/90 to PLGA 90/10. In one embodiment, PLGA 75/25 or PLGA 85/15 is used for manufacturing the pharmaceutical preparation of the invention.

Typically, the loading efficiency, or the encapsulation ratio of hFIX in the microspheres is at least 80% by total mass. The loading dosage varies in density for different purposes, and is dependent on the dosing regimen which is commonly based on the patients' individual pharmacokinetics. The encapsulation ratio may be adjusted by anyone skilled in the art according to standard technologies.

The term “an effective amount” as used herein refers to an amount that is required to confer the intended therapeutic effect in the subject to be administrated. Effective amounts may vary, as recognized by those skilled in the art, depending on routes of administration, excipient usages, and the possibilities of co-usage with other agents. Preferably, the pharmaceutical preparation can be administered intravenously, intramuscularly or subcutaneously.

The pharmaceutical preparation may further comprise one or more pharmaceutically acceptable carriers, which may be dissolved in an aqueous solution. Such carriers include, but are not limit to: saline, buffered saline, dextrose, water, glycerol, ethanol and a combination thereof.

The present invention also provides a method for manufacturing the pharmaceutical preparation of the present invention, comprising:
a) mixing a therapeutically effective amount of a human Factor IX (hFIX) in an aqueous solution with a lipophilic biodegradable polymer or copolymer in an organic solution to obtain a primary emulsion;
b) mixing the primary emulsion with a surfactant solution to obtain a secondary emulsion; and
c) evaporating the organic solvent, then filtrating, washing, and lyophilizing the secondary emulsion to obtain a powder-like pharmaceutical preparation;
whereby in the powder-like pharmaceutical preparation, the hFIX is encapsulated by the lipophilic biodegradable polymer or copolymer to form a microsphere which has an efficacy of sustained release of hFIX and prolonged biological activity.

According to the invention, the microsphere is prepared in the form of water-in-oil-in-water (W/O/W) emulsion. The hFIX is dissolved in the first aqueous solution at the concentration of 0.001% to 90% by mass based on that of the first aqueous solution. In one embodiment of the present invention, the weight of the hFIX is 20 mg. The first aqueous solution containing hFIX is then mixed with an organic solution containing at least one lipophilic biodegradable polymer or copolymer in a ratio of 1:1 to 1:100 (v:v) to obtain a primary emulsion. In one embodiment of the invention, the aqueous solution and the organic solution are in a ratio of 1:10.

According to the present invention, the organic solvent for preparing an organic solution is selected from the group consisting of dicholoromethane, chloroform, ethyl acetate, 1,4-dioxane, dimethyl formamide (DMF), dimethyl sulfoxide (DMSO), toluene, and tetrahydrofuran (THF). In one embodiment of the invention, the organic solvent is dicholoromethane.

Optionally, the solution used to prepare the primary emulsion may further contain at least one surfactant suitable for the production of the primary emulsion. In one embodiment, the one or more surfactant is selected from the group consisting of a glycerin fatty acid ester, a sucrose fatty acid ester, a sorbitan fatty acid ester, a propylene glycol fatty acid ester, diacetyl tartaric acid esters of mono-and diglycerides, a sodium aluminum phosphate, polysorbate 20, polysorbate 60, polysorbate 65, polysorbate 80, a hydroxypropyl cellulose, a hydroxylpropyl methylcellulose, mono-and diglycerides citrated, mono-and diglycerides tartrated, mono-and diglycerides lactated, mono-and diglycerides ethoxylated, mono-and diglycerides monosodium phosphate derivatives, succinylated monoglycerides, polyglycerol esters of fatty acid, polyglycerol esters of interesterified ricinoleic acids, calcium stearyl-2-lactylate, salts of fatty acids, a polyoxyethylene(20) sorbitan monopalmitate, polysorbate 40, polyoxyethylene(20) monostearate, a polyoxyethylene(20) sorbitan tristearate, Triton x-100, Tween 40, polyethylene glycol 200-800, a sodium lauryl sulfate, alcohol ethoxylates, alkylphenol ethoxylates, alkyl polyglycosides, and a combination thereof.

According to the invention, the primary emulsion is mixed with a surfactant solution to obtain a secondary emulsion. In one embodiment of the present invention, the surfactant may be, but are not limit to a glycerin fatty acid ester, a sucrose fatty acid ester, a sorbitan fatty acid ester, a propylene glycol fatty acid ester, diacetyl tartaric acid esters of mono-and diglycerides, a sodium aluminum phosphate, polysorbate 20, polysorbate 60, polysorbate 65, polysorbate 80, a hydroxypropyl cellulose, a hydroxylpropyl methylcellulose, mono-and diglycerides citrated, mono-and diglycerides tartrated, mono-and diglycerides lactated, mono-and diglycerides ethoxylated, mono-and diglycerides monosodium phosphate derivatives, succinylated monoglycerides, polyglycerol esters of fatty acid, polyglycerol esters of interesterified ricinoleic acids, calcium stearyl-2-lactylate, salts of fatty acids, a polyoxyethylene(20) sorbitan monopalmitate, polysorbate 40, polyoxyethylene(20) monostearate, a polyoxyethylene(20) sorbitan tristearate, Triton x-100, Tween 40, polyethylene glycol 200-800, a sodium lauryl sulfate, alcohol ethoxylates, alkylphenol ethoxylates, alkyl polyglycosides, or a combination thereof. In one embodiment of the invention, the surfactant is PVA, for example at a concentration of approximately 0.1% to 1.0% in the second aqueous solution, preferably 0.5%.

According to the present invention, the secondary emulsion is obtained from the surfactant solution and the primary emulsion in a ratio of 1:1 to 1000:1. In one example of the present invention, the ratio is 30:1. After evaporating the organic solvent from the secondary emulsion, the secondary emulsion is subsequently filtered, washed and lyophilized by conventional methods in the art to obtain a powder-like pharmaceutical preparation. The evaporation time may be set as from 0.1 to 24 hours. In one embodiment of the present invention, the evaporation time is preferably 3 to 4 hours.

To disperse two or more immiscible liquids, such as the emulsification of oil in water, ultrasonication may be performed through any known or commonly used methods or technologies. According to the present invention, ultrasonication may be performed at a power of 10 to 500 W for 0.01 to 30 min. In one preferred embodiment of the present invention, the ultrasonication is performed at a power of 75 W for 2-3 min. Any skilled person in the art would be able to choose the suitable condition according to different loading dosages or different materials used through repeated experiments. The present invention is further illustrated by the following examples, which are provided for the purpose of demonstration rather than limitation.

### EXAMPLE 1

### Preparation of the Sustained Release Microsphere of Encapsulated Human Recombinant Factor IX

Recombinant human factor IX (rhFIX) was obtained from milk of transgenic pig which was provided by Animal Technology Institute Taiwan (ATIT). After collecting the pig milk, it was centrifuged by 3,000×g at 4° C. for 20 minutes to remove fat. The fraction containing rhFIX was obtained from the defat milk by precipitation with a phosphate buffer solution and centrifugation by 22860×g, at 4° C. for 10 minutes. The whey fraction was further concentrated by ultrafiltration using a polysulfone membrane (TAMI) with a molecular cutoff of 30 kD. The rhFIX was subsequently captured and purified by Q Sepharose fast flow chromatography (Amersham Pharmacia Biotech) and Heparin-Sepharose column (Amersham Pharmacia Biotech). Nanofiltration was performed as the step of viral removal.

The rhFIX dissolved in distilled water and 90 mg/ml polylacto-glycolic acid (PLGA) dissolved in dichloromethane were mixed in a ratio of 1:10 (v:v), and then the mixture was ultrasonicated at 75 W for 2-3 min to obtain a primary emulsion. The primary emulsion was further mixed with 0.5% PVA in a ratio of 1:30 (v:v) and ultrasonicated at 75 W for 2-3 min to obtain a secondary emulsion. After the organic solvent was vaporized from the secondary emulsion for 3-4 hours, the secondary emulsion was filtrated, washed and lyophilized to obtain a PLGA microsphere encapsulated rhFIX. The filtered solution and cleaning liquid was collected during the procession, and combined as “combined solution” which was used for determining the encapsulation ratio and drug dosage.

### EXAMPLE 2

### Determining Release Kinetics and Activity of Encapsulated rhFIX by an in vitro Simulated Physiological Test

The simulated physiological test was performed by adding the sustained release microsphere prepared in Example 1 into a physiological buffer (0.5% Tween 20, PBS, pH7.4) maintained at 37° C. Small aliquots of the buffer were collected to determine the activity of rhFIX released from the microsphere every day. The rhFIX activity was determined by an aPTT (Activated Partial Thromboplastin Time) test and at the same time used FIX deficient plasma as a control.

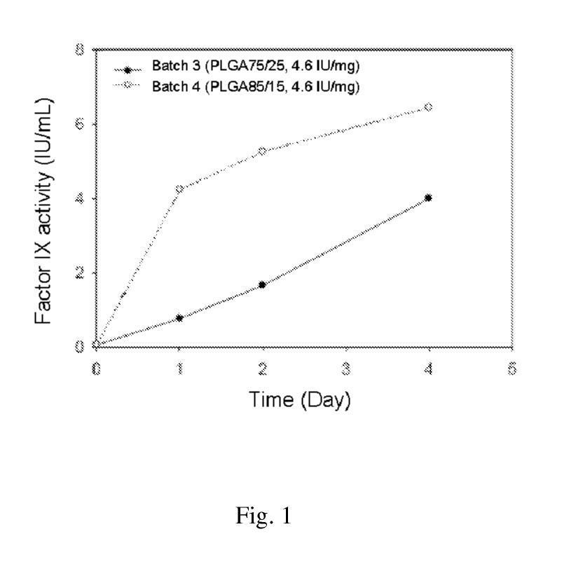

The aPTT test is a clotting time test which is used for determining FIX activity in a sample. Briefly, a fixed amount (25 p1) of plasma sample was mixed with 50 μl aPTT reagent and incubated at 37° C. for 1 min. Then, 50 μl 0.025 M CaCl₂ was added into the sample to initiate the analysis, which is performed at 37° C. for 4 min, under 660 nm by an automated blood clot coagulation analyzer (TECO Coatron M4), whose principle is to measure the change of light scattered from the sample as it clots. The results of the in vitro test are shown in FIG. 1.

As shown in FIG. 1, the rhFIX released from the preparations according to the present invention tested in vitro retained its activities, and the rhFIX gradually released from the preparations of PLGA85/15 (Batch 4) and PLGA 75/25 (Batch 3) for up to four days.

### EXAMPLE 3

### An in vivo Test on the Bioactivity of the rhFIX Released from the Microsphere

A hemophilia B mice model was used to determine the prolonged release of rhFIX from the preparation according to the present invention, compared to unencapsulated rhFIX. Six hemophilia B mice (R333Q-hFIX mice, gifts from Darrel W. Stafford, Department of Biology, UNC Chapel Hill, Chapel Hill) for each group were administered 50 IU/kg rhFIX for testing. Unencapsulated rhFIX and the preparation according to the present invention were administered intravenously through the tail vein and subcutaneously at the abdominal site, respectively. At different times after the administration, blood was collected from each mouse and centrifuged to obtain the plasma sample. The activity was determined by the aPTT assay as mentioned above. Blood clotting time which exceeds 120 seconds was deemed as weak or no activity of rhFIX. The detailed data was summarized in Table I. Data are presented as mean ±standard deviation, and statistical evaluation was performed by independent t test (SPSS version 12.0, Claritas Inc.). Value of p<0.05 was considered as statistically significant.

As shown in Table I, the effects of rhFIX on shortening blood coagulation time last for 48 hours (2 days) and became insignificant 72 hours (3days) after the administration in the group administered with the unencapsulated rhFIX. On the contrary, in the group administered with the preparation according to the present invention, the bioactivity of rhFIX was extended to at least 120 hours (5 days), and became insignificant until 168 hours (7 days) after the administration.

TABLE I

| | Blood Clotting Time (aPTT, sec.) | |
|---|---|---|
| Sampling Time (hr.) | Encapsulated rhFIX | Unencapsulated rhFIX |
| 0.25 | 37.40 ± 5.41 | 42.82 ± 2.74 (p = 0.078) |
| 24 | 41.87 ± 14.74 | 74.58 ± 8.29 (p < 0.01) |
| 48 | 51.40 ± 7.39 | 81.33 ± 3.45 (p < 0.01) |
| 72 | 58.63 ± 3.66 | 121.17 ± 16.93 (p < 0.01) |
| 96 | 68.37 ± 2.90 | >120 sec |
| 120 | 63.70 ± 5.74 | >120 sec |
| 168 | 92.73 ± 5.31 | — |

Given the above, it was concluded that the pharmaceutical preparation of the present invention provided a prolonged release of rhFIX, and the bioactivity of rhFIX in vivo maintained for at least 5 days. Therefore, the frequency of the injection may be decreased to 1 every 5 or 7 days, and it would significantly reduce the hemophilia B patient's burden. Furthermore, the preparation in a powder-like form generally provides a better chemical stability and could be stored for a longer period.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

1. A pharmaceutical preparation in a powder-like form, which is featured in comprising a therapeutically effective amount of a human Factor IX (hFIX), which is encapsulated by a lipophilic biodegradable polymer or copolymer to form a microsphere, whereby the pharmaceutical preparation provides a sustained release of hFIX and a prolonged biological activity, wherein the lipophilic biodegradable polymer or copolymer is selected from the group consisting of a phospholipid, a lecithin, a polyglycolic acid (PGA), a poly(lactic-co-glycolic acid) (PLGA), a poly(γ-glutamic acid), a polyvinylic acid (PVA), a γ-polyglutamic acid, a polycaprolactone, a polyanhydrides, a polyamino acid, a polydioxanone, a polyhydroxybutyrate, a polyphosphazenes, a polyesterurethane, a polycarboxsyphenoxypropane-cosebacic acid, a polyorthoester, and a combination thereof.

2. The pharmaceutical preparation of claim 1, which is featured in being used for treating hemophilia B.

3. The pharmaceutical preparation of claim 1, which is featured in that the lipophilic biodegradable polymer or copolymer is poly(lacto-glycolic) acid (PLGA).

4. The pharmaceutical preparation of claim 1, which is featured in that the microsphere has a diameter from 0.1 to 100 μm.

5. The pharmaceutical preparation of claim 1, which is featured in that the hFIX is in an amount of 0.0005 U/mg to 25 U/mg.

6. A method for manufacturing the pharmaceutical preparation of claim 1, which is featured in comprising:
a) mixing a therapeutically effective amount of a human Factor IX (hFIX) in an aqueous solution with a lipophilic biodegradable polymer or copolymer in an organic solution to obtain a primary emulsion;
b) mixing the primary emulsion with a surfactant solution to obtain a secondary emulsion; and
c) evaporating the organic solvent, then filtrating, washing, and lyophilizing the secondary emulsion to obtain a powder-like pharmaceutical preparation;
whereby in the power-like pharmaceutical preparation, the hFIX is encapsulated by the lipophilic biodegradable polymer or copolymer to form a microsphere so as to provide a sustained release of hFIX and a prolonged biological activity.

7. The method of claim 6, which is featured in that the solution of step (a) may further contain a surfactant.

8. The method of claim 6, which is featured in that the lipophilic biodegradable polymer or copolymer is selected from the group consisting of a phospholipid, a lecithin, a polyglycolic acid (PGA), a poly(lactic-co-glycolic acid) (PLGA), a poly(γ-glutamic acid), a polyvinylic acid (PVA), a γ-polyglutamic acid, a polycaprolactone, a polyanhydrides, a polyamino acid, a polydioxanone, a polyhydroxybutyrate, a polyphosphazenes, a polyesterurethane, a polycarboxsyphenoxypropane-cosebacic acid, a polyorthoester, and a combination thereof.

9. The method of claim 6, which is featured in that the lipophilic biodegradable polymer or copolymer is poly(lacto-glycolic) acid (PLGA).

10. The method of claim 6, which is featured in that the surfactant is selected from the group consisting of a glycerin fatty acid ester, a sucrose fatty acid ester, a sorbitan fatty acid ester, a propylene glycol fatty acid ester, diacetyl tartaric acid esters of mono-and diglycerides, a sodium aluminum phosphate, polysorbate 20, polysorbate 60, polysorbate 65, polysorbate 80, a hydroxypropyl cellulose, a hydroxylpropyl methylcellulose, mono-and diglycerides citrated, mono-and diglycerides tartrated, mono-and diglycerides lactated, mono-and diglycerides ethoxylated, mono-and diglycerides monosodium phosphate derivatives, succinylated monoglycerides, polyglycerol esters of fatty acid, polyglycerol esters of interesterified ricinoleic acids, calcium stearyl-2-lactylate, salts of fatty acids, a polyoxyethylene(20) sorbitan monopalmitate, polysorbate 40, polyoxyethylene(20) monostearate, a polyoxyethylene(20) sorbitan tristearate, Triton x-100, Tween 40, polyethylene glycol 200-800, a sodium lauryl sulfate, alcohol ethoxylates, alkylphenol ethoxylates, alkyl polyglycosides, and a combination thereof.

11. The method of claim 6, which is featured in that the organic solvent is selected from the group consisting of dicholoromethane, chloroform, ethyl acetate, 1,4-dioxane, dimethyl formamide (DMF), dimethyl sulfoxide (DMSO), toluene, tetrahydrofuran (THF), and a combination thereof.
